(19) United States
(12) Patent Application Publication
(10) Pub. No.: US 2023/0329313 A1
(43) Pub. Date: Oct. 19, 2023

(54) METHODS OF MAKING NON-AQUEOUS CHEWS

(71) Applicant: Florida Research Group, LLC, Tampa, FL (US)

(72) Inventor: Kotha SEKHARAM, Tampa, FL (US)

(21) Appl. No.: 18/302,171

(22) Filed: Apr. 18, 2023

Related U.S. Application Data
(60) Provisional application No. 63/332,499, filed on Apr. 19, 2022.

(51) Int. Cl. A61K 31/7008; A61K 9/00; A23L 29/262; A23K 20/121; A23K 20/158; A23P 30/10; A61K 47/38; A61K 47/36; A61K 31/122; A23L 29/10; A23L 29/00; A23K 40/20; A61K 47/26; A23K 20/163; A61K 31/737; A61K 47/46; A61K 47/12; A23P 20/20

(52) U.S. Cl. CPC A23P 30/10; A23K 20/121; A23K 20/158; A23K 20/163; A23K 40/20; A23L 29/04; A23L 29/10; A23L 29/262; A23P 20/20; A61K 9/0056; A61K 31/122; A61K 31/7008; A61K 31/737; A61K 47/12; A61K 47/26; A61K 47/36; A61K 47/38; A61K 47/46

(57) ABSTRACT

The invention relates to the field of orally administrable pharmaceutical and nutritional supplement dosage form; in particular, units in the form of an edible mass as chews for animals as well as for humans.

### CROSS REFERENCE TO A RELATED APPLICATION

This application claims the benefit of U.S. Provisional Application Serial No. 63/332,499, filed Apr. 19, 2022, which is incorporated herein by reference in its entirety.

### BACKGROUND OF THE INVENTION

Chews are normally made by blending dry ingredients with liquids to form a dough-like consistency. The materials are then shaped by passing through an extruder with a die or by using a dough molding machine where the dough is pushed into a molding cavity and then a bottom plate moves back to facilitate discharge. Water, glycerin, corn syrup, molasses, oils, fat and lecithin, are some of the liquid ingredients that are used; polyethylene glycol may be included in these chews to prevent sticking to dies.

Addition of water is avoided in making chews with pharmaceutical ingredients as the water accelerates degradation of the active ingredient. Other problems with usage of water are that pharmaceutical grade water is expensive, there is a possibility of microbial contamination, and water evaporates with time such that chews becomes brittle and lose the desired texture.

Chews made without adding water are called anhydrous or non-aqueous chews. Bound water, which inherently exists with most raw materials is also minimized by selecting the proper ingredients. Generally, these formulations depend on fats and oils.

US 9,314,478 teaches a method of making chews without adding water by mixing nutritional or pharmaceutically-active agents with a fat and extruding the composition at a temperature sufficient to melt the fat, and allowing the extruded composition to cool to room temperature thereby providing the chewable formulation.

US 8,114,455 teaches a method wherein dry ingredients of the chew mixture are blended first, followed by admixture with the liquid ingredients (e.g., humectants and softening agents) to form a thoroughly blended mixture. After blending, the chew mixture is formed into individual chunks using a molding machine with dies for production of chunk-like shapes, and packaged for storage.

US 9,532,946 teaches usage of a rotary molding machine for manufacturing of semi-plastic pharmaceutical dosage units.

Examples of chewable dosage forms may be found in US Pat Nos. 6,387,381; 4,284,652; 4,935,243; 6,270,790; 6,060,078; 4,609,543; and, 5753,255.

Almost all chews, including those mentioned above are based on preparing a shapable dough, and then shaping them by using an expensive extruder or molding machine. These machines have their own limitations, including the inability to shape dough with high density, limited scope to incorporate oil-based ingredients, need for additional ingredients such as polyethylene glycol or oils to prevent sticking of dough to mold cavities and the need for curing for about 24 to 48 hours before packing. All these factors cause extra cost and time. Every formulation works differently and hence needs individual attention.

There is a need for an improvement in the method of making non-aqueous chews without the need for expensive extruders or forming machines and free from limitations mentioned above.

### BRIEF SUMMARY OF THE INVENTION

The present invention provides improved non-aqueous chews, and methods of manufacturing the same, to deliver, for example, a nutritional or pharmaceutical active agent to animals as well as to humans. These improved chews can contain a nutritional ingredient or pharmaceutical active agent, or a treat with no specific functional ingredient.

Surprisingly, in accordance with the present invention it has been discovered that non-aqueous chews can be made:
(1) without adding water,
(2) without the need to make time-consuming dough-based system,
(3) without the need for any specialized equipment such as extruders and forming machines, and
(4) without the need for long curing times.

In specific embodiments, the subject invention provides:

A process of manufacturing edible, non-aqueous chews without a need of an extruder or chew molding machine comprising:
a) blending ethyl cellulose, oleaginous composition, and polyoxyethylene sorbitan fatty acid ester,
b) heating together to a temperature not less than 140° C. for at least 5 minutes,
c) blending with excipients, and optional active ingredients to form a pourable liquid, and
d) such liquid is poured into a mold of predetermined shape, size and volume, and upon cooling to room temperature, the liquid solidifies to form non-aqueous chews, or
   d′) such liquid is poured in a flat tray of required depth and upon cooling to room temperature, the liquid solidifies as a thick layer; and as an option the above steps are repeated to pour multiple layers, one on top of the other having different colors and textures and when solidified, the resulting product is cut to desired shape and size for packaging, or
   d″) such liquid is poured as a controlled flow into a cooling tub with food grade non-oil cooling medium and up on contact with cooling medium the liquid solidifies and forms natural looking flakes, or
   d‴) such liquid is poured in a flat tray of desirable depth and up on cooling to room temperature, the liquid solidifies as a thick layer; and such thickened product is comminuted, blended with granular excipients to compress in a pellet mill or a tablet press to form as pellets or wafers.

### DETAILED DESCRIPTION OF THE INVENTION

### Materials

All inactive materials used in making chews for human as well animal use are edible and of food grade or pharmaceutical grade. Those of ordinary skill in the art are familiar with such inactive ingredients, which need not include water for use in the invention.

Ethyl cellulose, an ethyl ether of cellulose, is a long-chain polymer of b-anhydro glucose. Ethyl cellulose, a GRAS-listed inactive ingredient is widely used in oral and topical pharmaceutical formulations, as a coating agent, flavoring fixative, tablet binder, tablet filler and viscosity-increasing agent. It is a tasteless, free-flowing, white to light tan colored powder. Ethyl cellulose with ethoxyl substitution of 44% to 51% and viscosity of about 90 to 110 cP are preferred.

Polyoxyethylene sorbitan fatty acid esters (polysorbates) are a series of partial fatty acid esters of sorbitol and its anhydrides copolymerized with approximately 20, 5, or 4 moles of ethylene oxide for each mole of sorbitol and its anhydrides. Polyoxyethylene sorbitan fatty acid esters are used as emulsifying agents; nonionic surfactants; solubilizing agents; wetting, dispersing/ suspending agents. Polysorbates 20, 40, 60, and 80 are included in the FDA Inactive Ingredients Guide and are accepted as food additives in Europe.

Stearic acid as a mixture of stearic acid (C18H36O2) and palmitic acid (C16H32O2). Stearic acid is widely used in oral and topical pharmaceutical formulations as an emulsifying agent, solubilizing agent, tablet and capsule lubricant.

### EXAMPLES

A greater understanding of the present invention and of its many advantages may be had from the following examples, given by way of illustration. The following examples are illustrative of some of the methods, applications, embodiments, and variants of the present invention. They are not to be considered as limiting the invention. All percentages are by weight and all solvent mixture proportions are by volume unless otherwise noted. Numerous changes and modifications can be made with respect to the invention.

### Example 1: Process for Manufacturing Non-Aqueous Chews

An example of non-aqueous chews made without using extruder is set forth in Formula 1 below:

Formula 1

| Ingredient | Concentration % w/w |
|---|---|
| Ethyl cellulose | 8.0 |
| Polysorbate-80 | 2.0 |
| Vegetable oil | 68.0 |
| Stearic acid | 4.0 |
| Glucosamine HCl | 5.0 |
| Chondroitin | 5.0 |
| Cellulose fiber | 8.0 |

A 4 gram finished chew delivers 200 mg Glucosamine and 200 mg Chondroitin.

### Example 2: Non-Aqueous Bi-Layer Chews

An example of non-aqueous bi-layer chews made without using extruder is set forth in Formula 2 below:

Formula 2

| Ingredient | Concentration % w/w |
|---|---|
| First layer (no color) | |
| Ethyl cellulose | 8.00 |
| Polysorbate-80 | 2.00 |
| Vegetable oil | 70.00 |
| Stearic acid | 4.00 |
| Rice bran | 8.00 |
| Starch | 8.00 |
| Ethyl cellulose | 8.00 |
| Polysorbate-80 | 2.00 |
| Vegetable oil | 69.92 |
| Stearic acid | 4.00 |
| Cellulose fiber | 8.00 |
| Starch | 8.00 |
| Astaxanthin | 0.08 |

### Example 3: Natural-Looking Non-Aqueous Flake Chews

An example of natural-looking non-aqueous flake chews made without using extruder is set forth in Formula 3 below:

Formula 3

| Ingredient | Concentration % w/w |
|---|---|
| Ethyl cellulose | 6.00 |
| Polysorbate-80 | 2.00 |
| Vegetable oil | 64.00 |
| Stearic acid | 8.00 |
| Chicken powder | 15.00 |
| Pork liver powder | 5.00 |

### Example 4: Non-Aqueous Chews With High Oil Content as Pellets and Kibbles

An example of non-aqueous chews with high oil content as pellets and kibbles made without using extruder is set forth in Formula 4 below:

Formula 4

| Base formula | |
|---|---|
| Ingredient | Concentration % w/w |
| Ethyl cellulose | 8.00 |
| Polysorbate-80 | 2.00 |
| Vegetable oil | 70.00 |
| Stearic acid | 4.00 |
| Cellulose fiber | 8.00 |
| Potato starch | 8.00 |
| Base formula | 50.00 |
| Roughage | 15.00 |
| Wheat by-product | 20.00 |
| Soy bean meal | 10.00 |
| Molasses | 5.00 |

### Example 5: Methods of Manufacture for Non-Aqueous Chews of the Invention

A. All dry ingredients listed in above Example 1 were sifted through a 20 mesh screen. Ethyl cellulose, polysorbate and stearic acid were added to oil and heated to 140⁰ C and maintained for at least 5 minutes. Other powdered ingredients and astaxanthin color, if present are added to the prepared oil mix. The mix is cooled until it is pourable and filled cavity molds or layered on a tray and set to cool at room temperature. When it is set, the finished product is removed from the mold. When the product is set in a tray, it is removed and cut to desired sizes.

B. To prepare bi-layered chews as in above Example 2, first, a layer without color was prepared by pouring in a tray. When the product is cooled and set, another layer with color is poured on the same tray and let it set. Prepared bilayer sheets were cut to desired measurements.

C. Example 3 is to prepare natural looking flakes or pellets. All dry ingredients listed in Example 3 were sifted through a 20 mesh screen. Ethyl cellulose, polysorbate and stearic acid were added to oil and heated to 140° C. and maintained for at least 5 minutes. Other powdered ingredients are added to prepared oil mix. The mix is cooled until it is pourable and the minx is slowly dropped in a tub with cooling medium. As soon as the liquid touches cooling medium, it solidifies. The product is collected and spread to separate any adhering cooling medium. Flow thickness, and flow rates decide on the shape of the finished product. Finished product looks like natural flakes.

D. All dry ingredients listed in Example 4 were sifted through a 20 mesh screen. Ethyl cellulose, polysorbate and stearic acid were added to oil and heated to 140° C. and maintained for at least 5 minutes. Other powdered ingredients are added stirring to prepared oil mix. The mix is cooled until it is in pourable nature and poured in a tray and set to cool to room temperature. When the product is set in a tray, it is removed and comminuted to fine flakes. These flakes were added to granular ingredients and passed through pellet mill or a tablet press to form pellets or wafers of required size and are packaged.

All patents, patent applications, provisional applications, and publications referred to or cited herein are incorporated by reference in their entirety, including all figures and tables, to the extent they are not inconsistent with the explicit teachings of this specification.

I claim:

1. A process of manufacturing edible, non-aqueous chews without a need of an extruder or chew molding machine comprising:
a) blending ethyl cellulose, oleaginous composition, and polyoxyethylene sorbitan fatty acid ester,
b) heating together to a temperature not less than 140° C. for at least 5 minutes,
c) blending with excipients, and optional active ingredients to form a pourable liquid, and
d) such liquid is poured into a mold of predetermined shape, size and volume, and upon cooling to room temperature, the liquid solidifies to form non-aqueous chews, or
d′) such liquid is poured in a flat tray of required depth and upon cooling to room temperature, the liquid solidifies as a thick layer; and as an option the above steps are repeated to pour multiple layers, one on top of the other having different colors and textures and when solidified, the resulting product is cut to desired shape and size for packaging, or
d″) such liquid is poured as a controlled flow into a cooling tub with food grade non-oil cooling medium and up on contact with cooling medium the liquid solidifies and forms natural looking flakes, or
d‴) such liquid is poured in a flat tray of desirable depth and up on cooling to room temperature, the liquid solidifies as a thick layer; and such thickened product is comminuted, blended with granular excipients to compress in a pellet mill or a tablet press to form as pellets or wafers.

2. The process according to claim 1 wherein ethyl cellulose has ethoxyl substitution of 44% to 51%.

3. The process according to claim 1 wherein ethyl cellulose has viscosity of about 50 cP to about 350 cP.

4. The process according to claim 1, wherein ethyl cellulose is about 0.5 wt % to 15 wt % of the finished product.

5. The process according to claim 1, wherein oleaginous composition is selected from the group consisting of soybean oil, canola oil, corn oil, sunflower oil, safflower oil, flaxseed oil, almond oil, peanut oil, fish oil, algal oil, palm oil, palm stearin, palm olein, palm kernel oil, high oleic soybean/canola/sunflower/safflower oils, hydrogenated palm kernel oil, hydrogenated palm stearin, fully hydrogenated soybean/canola/cottonseed oils, high stearic sunflower oil, enzymatically and chemically interesterified oils, butteroil, cocoa butter, avocado oil, almond oil, coconut oil, cottonseed oil, stearic acid, animal fat, cetyl myristoleate, lard, and chicken fat.

6. The process according to claim 1, wherein the oleaginous composition is hydrogenated palm oil.

7. The process according to claim 1, wherein the oleaginous composition is about 30 wt % to about 80 wt % of the finished product.

8. The process according to claim 1, wherein polyoxyethylene sorbitan fatty acid ester, is selected from the group consisting of polyoxyethylene sorbitan monooleate, polyoxyethylene sorbitan monostearate, sorbitan monooleate, sorbitan monostearate glyceryl monooleate, glyceryl monostearate, glyceryl monopalmitate, polyglyceryl ester of lauric acid polyglyceryl polylaurate, polyglyceryl ester of stearic acidpolyglyeryl polystearate, polyglyceryl ester of oleic acid, polyglyceryl polyoleate, polyglyceryl ester of ricinoleic acid, and polyglyceryl polyricinoleate.

9. The process according to claim 1, wherein the polyoxyethylene sorbitan fatty acid ester, is polysorbate-80.

10. The process according to claim 1, wherein the polyoxyethylene sorbitan fatty acid ester is about 1 wt % to about 15 wt % of the finished product.

11. The process according to claim 1, wherein comprising one or more excipients selects from sugar, bran, cereal, pulse, vegetables, herbs, starches, modified starches, salt, flour, protein, fiber, flavor, and color.

12. The process according to claim 1, wherein the excipient is about 10 wt % to about 50 wt % of the finished product.

13. The process according to claim 1, wherein the active ingredient is a therapeutically effective amount of a pharmaceutical ingredient, or a nutritional functional ingredient.
